(19) United States
(12) Patent Application Publication
(10) Pub. No.: US 2025/0032773 A1
(43) Pub. Date: Jan. 30, 2025

(54) METHOD AND DEVICE FOR DETECTING A WEAR CONDITION OF A VENTRICULAR ASSIST DEVICE AND FOR OPERATING SAME, AND VENTRICULAR ASSIST DEVICE

(71) Applicant: Kardion GmbH, Stuttgart (DE)

(72) Inventors: Hardy Baumbach, Stuttgart (DE); Julian Kassel, Böblingen (DE); Inga Schellenberg, Stuttgart (DE); Ricardo Ehrenpfordt, Korntal-Münchingen (DE); Marc Schmid, Stuttgart (DE); Ahmad Mansour, Weil der Stadt (DE); Martina Budde, Karlsruhe (DE); Thomas Alexander Schlebusch, Renningen (DE)

(21) Appl. No.: 18/917,558

(22) Filed: Oct. 16, 2024

Related U.S. Application Data
(63) Continuation of application No. 17/252,498, filed on Jul. 27, 2021, now Pat. No. 12,144,976.

(30) Foreign Application Priority Data
Jun. 21, 2018 (DE) 102018210076.4

(51) Int. Cl. A61M 60/816; A61M 60/174; A61M 60/178; A61M 60/216; A61M 60/237; A61M 60/538; A61M 60/546; A61M 60/554; A61M 60/592; A61M 60/857; G16H 40/40; G16H 40/67; G16H 50/70

(52) U.S. Cl. CPC A61M 60/816; A61M 60/174; A61M 60/178; A61M 60/216; A61M 60/237; A61M 60/538; A61M 60/546; A61M 60/554; A61M 60/592; A61M 60/857; G16H 40/40; G16H 40/67; G16H 50/70

(57) ABSTRACT

The present disclosure relates to a method for detecting a state of wear of a cardiac support system. The method comprises a read-in step and a determination step. During the read-in step, a sensor signal representing an operating state of the cardiac support system is read in. During the determination step, a wear signal is determined using the sensor signal and a comparison rule. The wear signal represents the wear condition.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application is a continuation of U.S. patent application Ser. No. 17/252,498, entitled METHOD AND DEVICE FOR DETECTING A WEAR CONDITION OF A VENTRICULAR ASSIST DEVICE AND FOR OPERATING SAME, AND VENTRICULAR ASSIST DEVICE, and filed Jul. 27, 2021, which is the U.S. National Phase of international application number PCT/EP2019/066486, entitled METHOD AND DEVICE FOR DETECTING A WEAR CONDITION OF A VENTRICULAR ASSIST DEVICE AND FOR OPERATING SAME, AND VENTRICULAR ASSIST DEVICE, and filed Jun. 19, 2019, the disclosure of which is hereby incorporated by reference herein in its entirety for all purposes and forms a part of this specification.

### BACKGROUND

### Field

The invention relates to a device or a method related to detecting a state of wear of a cardiac support system.

### Description of the Related Art

U.S. Pat. No. 5,904,708A describes a system with an implanted barometric pressure sensor, an extracorporeal barometric pressure sensor and an implanted processing unit that receives extracorporeal pressure and processes it to detect blood pressure changes. This system makes it possible to set or control a function of an implantable medical device using data from the implanted pressure sensor and the extracorporeal pressure sensor.

### SUMMARY

Based on this, the underlying object of the invention is to further improve the methods and devices known in the state of the art for operating and in particular for monitoring the condition of a cardiac support system as well as a cardiac support system as such and to ensure high functional reliability and ideally avoid critical system failures.

With this in mind, the approach presented here introduces a method for detecting a state of wear of a cardiac support system, a method for operating a cardiac support system as well as a device that uses and/or controls said method and a cardiac support system described herein. Advantageous further developments and improvements are described herein.

Using the approach presented here, a sensor signal representing an operating state of a cardiac support system can be read and processed. The operating state can be a physical state of the cardiac support system, for example, or a state of an environment in which the cardiac support system is being operated. Using the sensor signal and a processing specification, a wear signal representing a state of wear of the cardiac support system can be determined, for example by comparing the sensor signal with a specific operating state of the cardiac support system. This advantageously makes it possible to continuously monitor the condition of the cardiac support system, for example in order to detect damage or aging of the cardiac support system or a wear process of the cardiac support system. This is useful for being able to avoid a failure of the cardiac support system or being able to take action quickly in the event of such a state of wear. The safety and reliability of the cardiac support system can advantageously be increased by monitoring the condition and detecting the state of wear.

The invention presents a method for detecting a state of wear of a cardiac support system. The method comprises a read-in step and a determination step. In the read-in step, a sensor signal representing an operating state of the cardiac support system is read-in. In the determination step, a wear signal is determined using the sensor signal and a comparison rule. The wear signal represents the state of wear.

The state of wear can be understood to be a current condition of the cardiac support system or of components of the cardiac support system, such as a rotor, a sliding bearing, an impeller or a cable, which can affect a function of the cardiac support system. In the case of the impeller, for example, this could be an imbalance of said impeller. The state of wear can also be understood to be a property for example, such as a position or location of the cardiac support system or a component of the cardiac support system, such as a pump inlet, for example to be able to detect whether the pump inlet has suctioned itself to a blood vessel. The cardiac support system can, for example, be a right ventricular support system or a left ventricular support system which can, for example, be designed as an apical cardiac support system or as a cardiac support system for an aortic valve position. The sensor signal can be an electrical signal or a radio signal, which can be provided by a sensor device of the cardiac support system, for example. The sensor device can comprise a temperature sensor, a pressure sensor or a voltage sensor, for example, and the sensor signal can accordingly represent a temperature, a pressure or a pressure change, or a voltage as the operating state. It is also possible for the sensor signal to include data from a plurality of sensors, for example. In the determination step, a sensor parameter set, for example, can be extracted using the sensor signal. To evaluate the sensor parameter set, the wear signal can be determined using the sensor parameter set. The wear signal can be an electrical signal which, as the state of wear, for example comprises a deviation of the state of wear from a predefined initial state of the cardiac support system that is stored in the form of the processing specification.

According to one embodiment, at least one wear parameter can be determined in the determination step. The wear signal can include the wear parameter. The wear parameter can comprise technical data relating to a component of the cardiac support system, for example, from which the condition of the component can be inferred. The operating state reflected by means of the sensor signal can be evaluated in terms of a temporal progression, for example. The wear parameter can advantageously be used to analyze the state of wear, for example to be able to predict a time of failure for the cardiac support system or a component of the cardiac support system, which increases safety. It is also possible to ascertain the state of wear of a specific component, for example, so as to be able to replace the relevant component instead of replacing the entire cardiac support system, which is advantageously resource- and cost-saving.

Additionally or alternatively, according to one embodiment, at least one functional parameter representing a functionality of the cardiac support system can be determined in the determination step. In this case, the wear signal may include the functional parameter. The functional parameter can indicate whether further reliable operation of the cardiac support system is possible, for example, or whether replacement or maintenance is necessary. The functionality of the cardiac support system can thus advantageously be monitored particularly easily. An impairment of the functionality of the cardiac support system or a desired functionality of the cardiac support system can therefore be detected, for example to avoid maintenance of an implanted cardiac support system or to be able to plan said maintenance ahead of time, which advantageously increases the safety and reliability of the cardiac support system.

According to one embodiment, the method can furthermore comprise a step of providing the sensor signal, and additionally or alternatively the wear signal, to an interface with an external processing device. The external processing device can be an extracorporeal control device, for example, or a portable device such as a smartphone, or a server or a warning device for the cardiac support system. The operating state and additionally or alternatively the state of wear can thus advantageously be stored externally, for example for further evaluation. For provision to an interface with the warning device or to the smartphone, the wear signal can include the functional parameter, for example, and for provision to an interface with the extracorporeal control device or the server, the wear signal can include the wear parameter, for example. The wear signal can additionally also include the functional parameter and the wear parameter, for example for provision to an interface with a smartphone, for example to visually or audibly display the wear signal. The wear signal can additionally or alternatively be determined for provision as a warning signal using a predefined limit value as a processing specification and as a function of a comparison result with said limit value, for example.

According to one embodiment, the method can also comprise a sensing step. In the sensing step, the operating state is sensed and the sensor signal representing the operating state is provided. For this purpose, a specific component or a specific region of the cardiac support system can be sensed, for example by means of a defined sensor region, in order to ascertain the operating state. A sensor device for acquiring the sensor signal can thus advantageously be set, for example to determine the state of wear of a component of the cardiac support system, or to determine the state of wear particularly accurately.

According to one embodiment, the sensor signal can also be sensed using a sensor device. The sensor device can be configured to sense an electrical quantity, a temperature, a pressure, a volume flow, a movement, an optical or acoustic signal, a force, or a change in position of the cardiac support system. A voltage can be sensed as the electrical quantity, for example. A pressure build-up or a pressure difference can be sensed as the pressure, for example. A vibration of a component of the cardiac support system can be sensed as a movement, for example, or a sudden movement in the form of a fall or a physical impact on a wearer of an implanted cardiac support system. Sensing the pressure and the volume flow is advantageous, for example, to ascertain whether the pump inlet of the cardiac support system has suctioned itself to a blood vessel. A displacement or malfunction of the cardiac support system, such as an imbalance of the impeller, can be identified with the aid of an optical or acoustic signal that can be acquired by a laser interferometer, a microphone or a structure-borne sound sensor, for example. To sense the mentioned quantities, the sensor device can, for example, comprise a voltage sensor, an electrical resistance sensor, a temperature sensor, a pressure sensor, an ultrasonic flow sensor, an optical reflection coefficient sensor, a movement sensor, an acceleration sensor, a magnetic sensor, a microphone, a force sensor, a distance sensor or an inductive and/or capacitive rotor bearing sensor or a combination of said sensors. At least one of said sensors can additionally also be configured to be redundant, for example for self-diagnosis of the sensors. Sensors integrated into the cardiac support system can advantageously be used to provide the sensor signal. This is cost-saving and makes a compact design of a device for carrying out this embodiment of the method possible.

According to one embodiment, the sensor signal can also represent the operating state in the time domain and additionally or alternatively in the frequency domain. For this purpose, the sensor signal can represent the operating state within a specific period of time, for example in order to ascertain an average value or a standard deviation of the operating state. If the sensor signal represents the operating state in the frequency domain, characteristic frequencies of a median frequency of the spectrum, the integrated band energy in defined frequency bands or also the absolute amplitude at the location of known damage frequencies can be determined, for example. A further analysis of the sensor signal is thus advantageously made possible, which is advantageous for an exact determination of the state of wear.

Furthermore, according to one embodiment, a further sensor signal representing a further operating state of the cardiac support system can be read-in in the read-in step. In the determination step, the wear signal can be determined using the sensor signal, the further sensor signal and the comparison rule. This is advantageous in order to be able to use a plurality of sensor signals to determine the state of wear.

In this case, according to one embodiment, a sensor parameter set can be extracted in the determination step using the sensor signal and the further sensor signal. The wear signal can be determined using the sensor parameter set and the comparison rule. Determining the sensor parameter set is advantageous for transforming the sensor data in the time or frequency domain.

According to one embodiment, the method can comprise a step of defining the comparison rule using the sensor signal. The sensor parameter set can be used to do this, for example. The sensor signal can be used to create a profile of the operating state, for example, or a so-called fingerprint of the system can be generated. This embodiment advantageously makes it possible to use a sensor signal that has already been read in to define the comparison rule; for example to set the comparison rule to a specific operating state, for example to an implanted state of the cardiac support system.

This approach further introduces a method for operating a cardiac support system. The method comprises the steps of one embodiment of the aforementioned method for detecting a state of wear of a cardiac support system and a step of providing a control signal for controlling a component of the cardiac support system. The control signal is output using the sensor signal or the wear signal. A sensor signal that is already required for the normal operation of the cardiac support system, i.e. a sensor signal that is required to perform a cardiac support function of the cardiac support system, can thus also be used to detect and optionally evaluate wear of the cardiac support system. According to this design example, a component and consequently a function of the cardiac support system can advantageously be adapted using the sensor signal and additionally or alternatively the wear signal, for example to reduce or prevent damage to a mechanical element of the cardiac support system, depending on the operating state or the state of wear. For example, the pump of the cardiac support system can be slowed using the sensor signal to prevent damage in the event of a detected impact, such as a fall, detected by an acceleration sensor or a movement sensor, for example.

The approach presented here further creates a device configured to carry out, control or implement the steps of a variant of a method for detecting a state of wear of a cardiac support system presented here or for operating a cardiac support system in corresponding devices. This design variant of the invention in the form of a device also makes it possible to achieve the underlying object of the invention quickly and efficiently.

For this purpose, the device can comprise at least one computing unit for processing signals or data, at least one memory unit for storing signals or data, at least one interface to a sensor or an actuator for inputting sensor signals from the sensor or for outputting data or control signals to the actuator, and/or at least one communication interface for inputting or outputting data embedded in a communication protocol. The computing unit can be a signal processor, a microcontroller or the like, for example, whereas the memory unit can be a flash memory, an EEPROM or a magnetic memory unit. The communication interface can be configured to input or output data in a wireless and/or wired manner, whereby a communication interface that can input or output wired data can, for example, input or output said data electrically or optically from or to a corresponding data transmission line.

In the present case, a device can be understood to be an electrical device that processes sensor signals and outputs control and/or data signals as a function of said sensor signals. The device can comprise an interface that can be hardware and/or software-based. In the case of a hardware-based configuration, the interfaces can be part of a so-called system ASIC, for example, which contains the various functions of the device. However, it is also possible for the interfaces to be separate, integrated circuits or consist at least in part of discrete components. In the case of a software-based configuration, the interfaces can be software modules that are, for example, provided on a microcontroller alongside other software modules.

Such a device can advantageously be part of a cardiac support system or integrated into a cardiac support system. For example, one embodiment of the device can be integrated into an already functional cardiac support system in order to be able to monitor the state of wear of the cardiac support system using sensor signals that are to be acquired anyway during the operation of the cardiac support system.

### BRIEF DESCRIPTION OF THE DRAWINGS

Design examples of the approach presented here are shown schematically in the drawings and explained in more detail in the following description. The figures show:

FIG. 1 a schematic illustration of a cardiac support system in aortic valve position according to one design example;

FIG. 2 a schematic illustration of an apical cardiac support system according to one design example;

FIG. 3 a schematic illustration of a device for detecting a state of wear of a cardiac support system according to one design example;

FIG. 4 a schematic illustration of a device for operating a cardiac support system according to one design example;

FIG. 5 a schematic illustration of an arrangement of sensors of a cardiac support system with a device for detecting a state of wear of a cardiac support system according to one design example; and

FIG. 6 a flow diagram of a method for detecting a state of wear of a cardiac support system and a method for operating a cardiac support system according to one design example.

In the following description of favorable design examples of the present invention, the same or similar reference signs are used for the elements shown in the various figures, which have a similar effect, whereby a repeated description of these elements is omitted.

### DETAILED DESCRIPTION

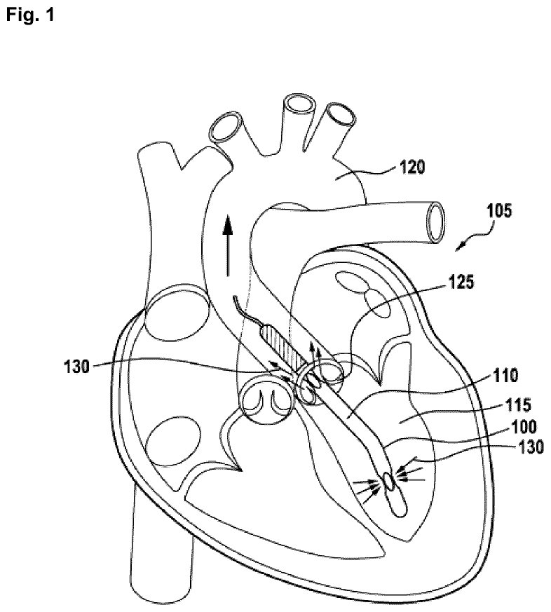

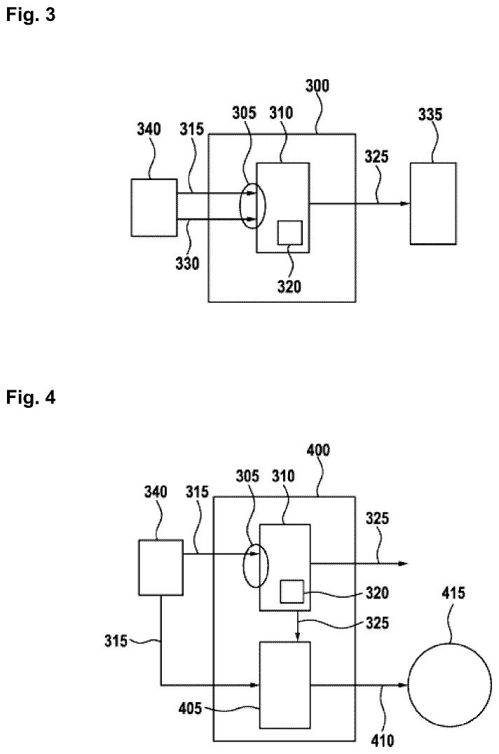

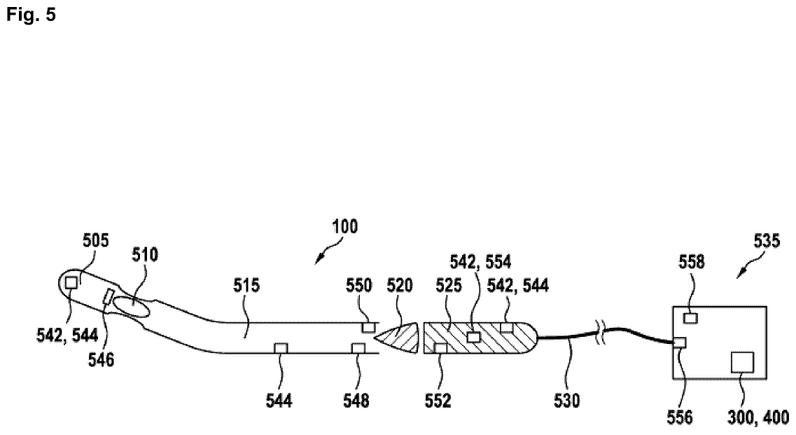

FIG. 1 shows a schematic illustration of a cardiac support system 100 in aortic valve position according to one design example. The figure shows a simple illustration of the cardiac support system 100 in the implanted state in a heart 105. In the aortic valve position of the cardiac support system 100 shown here, a section of the cardiac support system 100 with an inlet cannula 110 is disposed in the left ventricle 115 of the heart 105, and another section of the cardiac support system 100 is disposed in the aorta 120 in the region of the aortic valves 125. A pump volume flow 130 is received at the tip of the inlet cannula 110 in the ventricle 115 and discharged in the region of the aorta 120. One design example of the cardiac support system 100 shown here comprises a device for detecting a state of wear or for operating the cardiac support system 100, as shown with reference to FIGS. 3 and 4 described in the following.

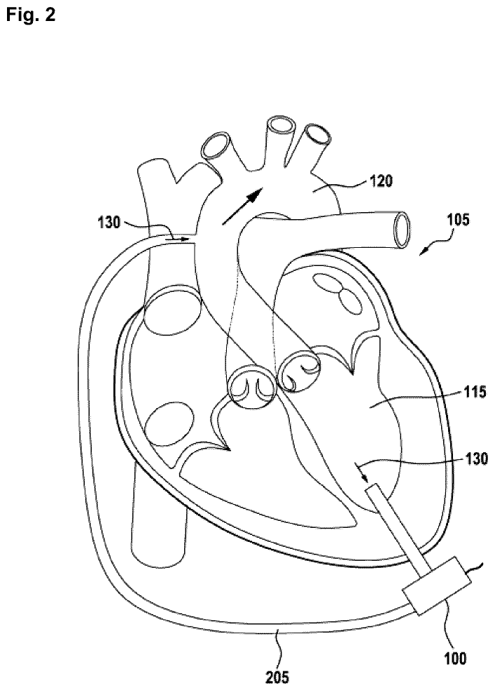

FIG. 2 shows a schematic illustration of an apical cardiac support system 100 according to one design example. The figure shows a simple illustration of the cardiac support system 100 in the implanted state. The apical cardiac support system 100 comprises an input for introducing a blood flow, which pumps a blood flow from the left ventricle 115 of the heart into the aorta 120 via a drain cannula 205 which is led along the heart 105 outside the heart 105. For this purpose, the pump volume flow 130 is delivered to the drain cannula 205 by a pump of the cardiac support system 100, for example a rotary pump. The drain cannula 205 delivers the pump volume flow 130 to the aorta 120. One design example of the apical cardiac support system 100 shown here also comprises a device for detecting a state of wear or for operating the cardiac support system 100, as shown with reference to FIGS. 3 and 4 described in the following.

FIG. 3 shows a schematic illustration of a device 300 for detecting a state of wear of a cardiac support system according to one design example. The device 300 comprises a reading device 305 and a determination device 310. The reading device 305 is configured to read in a sensor signal 315 that represents an operating state of the cardiac support system. The determination device 310 is configured to determine a wear signal 325 using the sensor signal 315 and a comparison rule 320. The wear signal 325 represents the state of wear.

The device 300 shown here can be used in conjunction with a cardiac support system such as one of the two cardiac support systems shown as an example in the preceding FIGS. 1 and 2.

According to one design example, the determination device 310 is configured to determine at least one wear parameter and to provide the wear signal 325 comprising the wear parameter. Additionally or alternatively, the determination device 310 is configured to determine at least one functional parameter representing a functionality of the cardiac support system and to provide the wear signal 325 comprising the functional parameter.

According to the design example shown here, the determination device 310 is also configured to provide the wear signal 325 to an interface with an external processing device 335. The sensor signal 315 is optionally also provided to the interface with the external processing device 335. The interface can be wireless or wired.

According to the design example shown here, the reading device 305 is also configured to read in at least one further sensor signal 330, which represents a further operating state of the cardiac support system. The determination device 310 is configured to determine the wear signal 325 using the sensor signal 315, the at least one further sensor signal 330 and the comparison rule 320. The determination device 310 is also optionally configured to extract a sensor parameter set using the sensor signal 315 and the at least one further sensor signal 330, and to determine the wear signal 325 using the sensor parameter set and the comparison rule 320. According to the design example shown here, the comparison rule is prestored in the determination device. The comparison rule 320 is optionally defined using the sensor signal 315.

According to the design example shown here, the sensor signal 315 and the further sensor signal 330 are provided by a sensor device 340. The sensor device 340 is optionally configured to sense the operating state and to provide the sensor signal 315 representing the operating state. According to one design example, the sensor device 340 is also configured to sense an electrical quantity, a temperature, a pressure, a volume flow, a movement, an optical or acoustic signal, a force, or a change in position of the cardiac support system in order to provide the sensor signal 315. According to one design example, the sensor signal 315 is configured to represent the operating state in the time domain and additionally or alternatively in the frequency domain.

In combination with a cardiac support system, the device 300 shown here can be used for monitoring at least one functional group of a cardiac support system so that a maintenance intervention can be carried out before the first symptoms or acute emergencies occur. The sensor device 340 can be a sensor device integrated into the cardiac support system, for example, and configured to determine operating parameters such as currents, voltages, temperatures, vibrations, pressures and pressure changes, sound, optical reflection coefficients, forces and changes in position. According to one design example, sensor parameters are extracted from the sensor data using the determination device 310, for example in the time domain and additionally or alternatively in the frequency domain.

A fingerprint of the system can, as it were, be generated from the determined sensor parameter set or the sensor parameter set can be regarded as such a fingerprint. Using the determination device 310, the fingerprint can continuously be compared to the definition of a healthy fingerprint in the form of the comparison rule 320. Deviations of the fingerprint from the healthy fingerprint of the comparison rule 320 are an indication of ongoing aging or damage processes of the cardiac support system. The temporal progression of the sensor parameter set is optionally employed using the determination device 310 to assess wear processes of the cardiac support system and possibly predict a time of failure. According to the design example shown here, the determination device 310 is configured to provide the wear signal 325 to the interface with the external processing device 335 to, in the event of a deviation from the normal fingerprint identified by means of the wear signal 325, for example in the form of the wear signal 325 comprising the wear parameter, inform the wearer of the implanted cardiac support system with the device 300, for example, the patient or a physician via the wear signal 325 representing the state of wear.

Such a monitoring of the condition of the cardiac support system using a variant of the device 300 shown here advantageously makes it possible to achieve a reduction or even a prevention of critical system failures. The early detection of a system degradation provides a time advantage, so that surgery appointments for component replacement, for example, can be planned early. Predictive maintenance interventions increases the patient's quality of life by not exposing the patient to a medical emergency scenario. The device 300 shown here can also be referred to as a condition monitoring system and provides an additional safeguard particularly for subsystems that cannot be configured to be redundant, such as the pump motor. By integrating the device 300 as a condition monitoring system, the patient's confidence in his support system can be increased, which results in a sense of security.

Using the wear signal 325, the determined state of wear can be transmitted via the interface with the external processing device 335 in the form of a communication interface, such as a radio modem or a wired interface. If the external processing device 335 comprises a display device, for example in the form of a screen of the extracorporeal control device or the portable device, a so-called “wearable”, such as a smartphone coupled via Bluetooth Low Energy as an external processing device 335, the state of wear provided by means of the wear signal 325 can be displayed on the display device, for example in the form of a condition measure of the state of wear.

By providing the wear signal 325 to the interface with the external processing device 335, the state of wear and/or the underlying sensor values or sensor parameters can additionally or alternatively also be stored for later retrieval (via cable, radio, or an inductively coupled communication interface) in the extracorporeal control device or a control device implanted with the cardiac support system and/or can be transmitted via a wide area communication network (for example, WLAN, LTE, or GPRS) to a central server. The use of a central server has the advantage that the system fingerprint and the parameter trend progression of the sensor data and the state of wear transmitted by means of the wear signal 325 can be compared to a large population of systems, so that it is possible to make robust statements about the system state of the cardiac support system.

FIG. 4 shows a schematic illustration of a device 400 for operating a cardiac support system according to one design example. The device 400 shown here is configured to operate and additionally or alternatively control the cardiac support system, such as the cardiac support system shown as an example with reference to FIG. 1 or FIG. 2. For this purpose, the device 400 comprises the reading device 305 and the determination device 310, which correspond substantially to the reading device and the determination device shown in FIG. 3. The reading device 305 is correspondingly configured to read in the sensor signal 315 provided by the sensor device 340, and the determination device 310 is configured to determine and provide the wear signal 325 using the sensor signal 315 and the comparison rule 320.

According to the design example shown here, the device 400 also comprises a control device 405. The control device 405 is configured to provide a control signal 410 for controlling a component 415 of the cardiac support system using the sensor signal 315 or the wear signal 325.

The component 415 of the cardiac support system is a control unit, for example, or a structural element such as a pump or an impeller or a drive device as shown in the following FIG. 5. If, by means of the sensor signal 315 and additionally or alternatively by the determination of the wear signal 325 using the determination device 310, the device 400 detects a fall or other physical impact on the patient, the device 400 is configured to, using the control device 405, control one of the components 415 of the cardiac support system, such as the pump, by means of the control signal 410 to temporarily slow or stop the pump in order to prevent or reduce damage to the mechanical elements.

FIG. 5 shows a schematic illustration of an arrangement of sensors of a cardiac support system 100 for a device 300 for detecting a state of wear of a cardiac support system 100 according to one design example. The example sensor integration into the cardiac support system 100 shown here can also be carried out in conjunction with the device 400 for operating the cardiac support system 100. The cardiac support system correspondingly comprises the device 300; 400, for example, which is similar to or the same as a variant of the device as described with reference to the preceding FIGS. 3 and 4. As an example, the cardiac support system 100 shown here is shown as a cardiac support system for the aortic valve position, like the cardiac support system described with reference to FIG. 1.

The cardiac support system 100 comprises a tip 505, an inlet cage 510 for receiving a blood volume flow, an inlet cannula 515 for delivering the blood volume flow to a micro-axial flow pump, an impeller 520 of the micro-axial flow pump, a magnetically or mechanically coupled electric drive 525, a supply cable 530 and a control unit 535. The control unit 535 comprises the device 300; 400, for example. As an example, the cardiac support system 100 comprises a variety of sensors as a sensor device in an example of a mounting position. According to the design example shown here, the cardiac support system 100 comprises three temperature sensors 542, two of which are disposed in the region of the electric drive 525 and one of which is disposed in the region of the tip 505. As an example, the cardiac support system 100 also comprises three pressure sensors 544, of which one is disposed in the region of the tip 505, one is disposed in the region of the inlet cannula 515 and one is disposed in the region of the electric drive 525. In the region between the tip 505 and the inlet cage 510, the cardiac support system also comprises an ultrasonic flow sensor 546. At an end facing away from the tip 505, the inlet cannula 515 additionally comprises a force, bending or distance sensor 548 and an optical reflection coefficient sensor 550. Adjacent to the impeller 520, the electric drive 525 comprises an impeller position sensor 552 in the form of a magnetic sensor or a Hall sensor, an optical distance sensor such as a laser interferometer, or an inductive and additionally or alternatively capacitive rotor position sensor. In the center of the electric drive 525, the cardiac support system 100 comprises a vibration sensor, a structure-borne sound sensor, a microphone and additionally or alternatively a microcontroller, for example in the form of a sensor hub, as a further impeller position sensor 554. In the region of the point of contact of the control unit 535 with the supply cable 530, the cardiac support system comprises a voltage sensor 556 in the form of a voltage, current, power, electrical resistance and/or back EMF sensor. The control unit 535 further comprises a control device sensor 558 in the form of a microphone, pressure, acceleration and/or rotation rate sensor, or temperature sensor.

A redundant design of the sensors 542, 544, 546, 548, 550, 552, 554, 556 and 558 shown here is advantageous for self-diagnosis of the sensors 542, 544, 546, 548, 550, 552, 554, 556 and 558 using deviations between the individual sensor values. An integration of all of the sensors 542, 544, 546, 548, 550, 552, 554, 556 and 558 shown here does not make sense in every application or, for reasons of installation space or cost, cannot be implemented in every application. The most relevant sensors can be selected in a targeted manner depending on the specific application, for example on the basis of a failure mode and effects analysis (FMEA analysis) or observed failures in long-term tests or stress tests.

The following is a list of application examples for the device 300, 400 in conjunction with the cardiac support system 100 and a sensor device such as one or more of the sensors 542, 544, 546, 548, 550, 552, 554, 556 and 558 shown here for detecting the state of wear of the cardiac support system 100 and/or for operating the cardiac support system 100:

The impeller sensor 554 in the form of the microphone or the structure-borne sound sensor and additionally or alternatively the control device sensor 558 in the form of the microphone and/or an acceleration sensor enables an analysis of the bearing wear by determining the state of wear.

In the case of magnetically coupled and magnetically mounted impellers 520, aging and deposits and the onset of pump thromboses can result in an imbalance of the impeller 520, which can be detected via the microphone, the acceleration sensor, the rotation rate sensor and the structure-borne sound sensor of the impeller sensor 554 and/or the control device sensor 558.

A change in friction in the sliding bearings of the cardiac support system 100, for example as a result of wear or the build-up of deposits, can be detected via a change in the power consumption, which can be sensed by a voltage sensor 556 in the form of a current, voltage, or power sensor, in combination with the actual pressure build-up or volume flow build-up, which can be sensed by one of the pressure sensors 544 in the region of the inlet cannula 515 or the electric drive 525 or the ultrasonic flow sensor 546. This error pattern furthermore also leads to a characteristic oscillation, which can be detected by the microphone, the acceleration sensor, the rotation rate sensor and the structure-borne sound sensor of the impeller sensor 554 and/or the control device sensor 558.

A measurement of the slippage between the magnetically coupled drive 525 and the impeller 520 provides information about the condition of the coupling and the sliding bearings of the impeller 520. The slippage can be sensed via an optical, magnetic or capacitive impeller position sensor 552, for example, or the phase relationship of the electric drive current and the back-induced field energy (back EMF) in currentless turns of the multiphase electric motor 525 can be sensed by means of the voltage sensor 556, for example in the control device 535 or in the electric drive 525. For this purpose, the voltage sensor 556 can be placed in the position shown here or in the region of the electric drive 525 in the position of the temperature sensor 542 or pressure sensor 554 disposed there.

A load on the bearings and a resulting pressure build-up of the impeller 520 can also be sensed at the bearing of the impeller by means of the force, strain or distance sensors 548.

Deposits and the onset of pump thromboses can be detected via a pressure drop in the inlet hose in the form of the inlet cannula 515, for example via pressure gradients between the aorta and the inlet cannula 515 or the ventricle, but also via a comparison of the electrical power consumption detectable by means of the voltage sensor 556 with the actual flow detectable by means of the ultrasonic flow sensor 546 and the pressure build-up of the pump detectable by means of the pressure sensors 544.

Indications of aging processes in the stator of the electric drive 525 are provided by the winding temperature that can be detected by means of the temperature sensor 542 positioned in the region of the electric drive 525, for example, or the winding impedance that can be detected by means of the voltage sensor 556, but also by optically, inductively or capacitively measured dimensions of the motor air gap measured by means of an impeller position sensor 552 in the position of the temperature sensor 542 or pressure sensor 544 disposed in the region of the electric drive 525.

Deposits on the rotor, as well as an imbalance, can also be determined via optical measurement of the reflection coefficient using the reflection coefficient sensor 550.

The quality of the supply cable 530 can be monitored via an electrical resistance measurement by means of the voltage sensor 556. In addition to detecting cable breaks (series measurement), the resistance measurement can also be carried out as a complex-valued impedance measurement between adjacent line strands to assess the condition of the insulation jacket and/or dielectric.

Faults in the power and signal electronics of the control device 535 can be detected by measuring the temperature of individual assemblies and monitoring selected voltage levels.

Suctioning of a pump inlet to the ventricular wall of the aorta in a cardiac support system 100 in aortic valve position, a so-called “suction”, can also be detected by means of the device 300, 400 shown here. A supporting blood volume flow is no longer possible if the cardiac support system 100 suctions on; the cardiac support system 100 should (automatically) reduce the pump power until said system releases from the aortic wall. Suctioning on can be detected via the pressure gradient of the pressure sensors in the region of the tip 505 and in the region of the inlet cannula 515 and by means of the ultrasonic flow sensor 546. Partial closure of the inlet cage 510 also changes the flow conditions in the inlet cage 510, which can be detected via the Doppler spectrum of the ultrasonic flow sensor 546.

The sensor data processing by means of the determination device of the device 300, 400 is based on the analysis of the sensor signals in the time domain, for example via relative or absolute threshold values, mean values, standard deviations, minimum and maximum values in time windows or the overall observation period. Additionally or alternatively, the sensor data processing by means of the determination device of the device 300, 400 is based on the analysis of the sensor signals in the frequency domain, for example via a determination of characteristic frequencies, a median frequency of the spectrum, the integrated band energy in defined frequency bands or also the absolute amplitude at the location of known damage frequencies. The mentioned sensor parameters of the operating state can be determined on the basis of predefined threshold values of the comparison rule as condition parameters, i.e. as wear parameters, for example. Alternatively, a fingerprint of the system parameters can also be defined as a comparison rule and, for example, a threshold value can be defined on a mathematical distance measure on the fingerprint defined as healthy, for example as a threshold value hyperplane in the multidimensional parameter space.

The processing of the sensor values can be realized in a pump-integrated microcontroller such as a sensor hub or in the control device 535. The sensor hub can also be used only for preprocessing the sensor data and forwarding extracted sensor parameters, which reduces the required communication bandwidth along the supply cable 530.

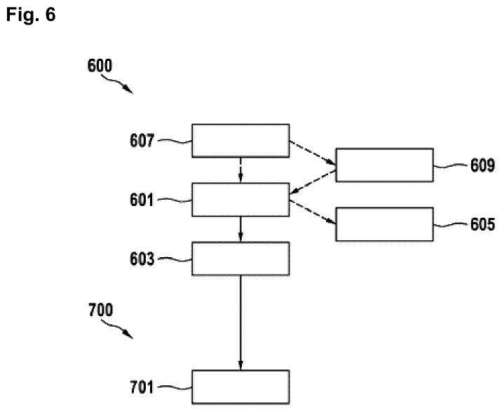

FIG. 6 shows a flow diagram of a method 600 for detecting a state of wear of a cardiac support system and a method 700 for operating a cardiac support system according to one design example.

The method 600 comprises a read-in step 601 and a determination step 603. In the read-in step 601, a sensor signal representing an operating state of the cardiac support system is read-in. In the determination step 603, a wear signal is determined using the sensor signal and a comparison rule. The wear signal represents the state of wear.

According to one design example, at least one wear parameter is determined in the determination step 603. In this case, the wear signal includes the at least one wear parameter. In the determination step 603, at least one functional parameter representing a functionality of the cardiac support system is optionally determined as well. The wear signal then comprises the at least one functional parameter.

The method 600 also optionally comprises a step 605 of providing the sensor signal and/or the wear signal to an interface with an external processing device. The provision step 605 optionally takes place after the determination step 603. If only the sensor signal is provided in the provision step 605, the provision step 605 can also take place subsequent to the read-in step 601.

According to one design example, the method 600 further comprises a sensing step 607, in which the operating state is sensed and the sensor signal representing the operating state is provided. The sensing step 607 optionally takes place before the read-in step 601. The sensing step 607 is additionally or alternatively carried out before the provision step 605.

In the read-in step 601, a further sensor signal representing a further operating state of the cardiac support system is optionally read-in. In this case, the wear signal is determined in the determination step 603 using the sensor signal, the further sensor signal and the comparison rule. A sensor parameter set is optionally also extracted in the determination step 603 using the sensor signal and the further sensor signal. The wear signal is then determined using the sensor parameter set and the comparison rule.

According to one design example, the method 600 further comprises a step 609 of defining the comparison rule using the sensor signal. The defining step 609 is optionally carried out after the read-in step 601 before the determination step 603.

The method 700 for operating a cardiac support system comprises at least step 601 and step 603 of the method 600 and optionally one or more of steps 605, 607 and 609 as described above. The method 700 further comprises a step 701 of providing a control signal for controlling a component of the cardiac support system. The control signal is provided using the sensor signal or the wear signal.

If a design example includes an “and/or” conjunction between a first feature and a second feature, this should be read to mean that the design example according to one embodiment comprises both the first feature and the second feature and, according to another embodiment, comprises either only the first feature or only the second feature.

1-13. (canceled)

14. A cardiac support system, the cardiac support system comprising:
an impeller;
a sensor associated with the impeller; and
a condition monitoring system configured to:
store a current fingerprint, the current fingerprint based at least in part on a sensor signal from the sensor of the cardiac support system and associated with an operating state of an impeller of the cardiac support system implanted into a heart;
compare the current fingerprint with a predetermined fingerprint based at least in part on a comparison rule;
determine a wear signal based at least in part on the comparison between the current fingerprint and the predetermined fingerprint;
determine a failure prediction associated with the cardiac support system based on the wear signal; and
cause an interface to provide an alert associated with the failure prediction.

15. The cardiac support system of claim 14, wherein the failure prediction includes a predicted time of failure of the cardiac support system.

16. The cardiac support system of claim 14, wherein the sensor signal is associated with deposits on the impeller or an imbalance of the impeller.

17. The cardiac support system of claim 14, wherein the sensor signal is associated with at least one of: position, vibration, acceleration, pressure, rotation rate, temperature, voltage, current, power, optical reflection, or electrical resistance associated with the impeller.

18. The cardiac support system of claim 14, wherein the sensor signal represents the operating state of the cardiac support system in the time domain or the frequency domain.

19. The cardiac support system of claim 14, the condition monitoring system further to:
provide the sensor signal or the wear signal to an external processing device.

20. The cardiac support system of claim 14, further comprising a control device configured to:
generate a control signal configured to control a component of the cardiac support system, the control signal generated based at least in part on the wear signal; and
transmit the control signal for controlling a component of the cardiac support system.

21. A method for monitoring a cardiac support system, the method comprising:
storing a current fingerprint, the current fingerprint based at least in part on a sensor signal from a sensor of the cardiac support system and associated with an operating state of an impeller of the cardiac support system implanted into a heart;
comparing the current fingerprint with a predetermined fingerprint based at least in part on a comparison rule;
determining a wear signal based at least in part on the comparison between the current fingerprint and the predetermined fingerprint;
determining a failure prediction associated with the cardiac support system based on the wear signal; and
causing an interface to provide an alert associated with the failure prediction.

22. The method of claim 21, wherein the failure prediction includes a predicted time of failure of the cardiac support system.

23. The method of claim 21, wherein the sensor signal is associated with deposits on the impeller or an imbalance of the impeller.

24. The method of claim 21, wherein the sensor signal is associated with at least one of: position, vibration, acceleration, pressure, rotation rate, temperature, voltage, current, power, optical reflection, or electrical resistance associated with the impeller.

25. The method of claim 21, wherein the sensor signal represents the operating state of the cardiac support system in the time domain or the frequency domain.

26. The method of claim 21, further comprising providing the sensor signal or the wear signal to an external processing device.

27. The method of claim 21, further comprising:
generating a control signal configured to control a component of the cardiac support system, the control signal generated based at least in part on the wear signal; and
transmitting the control signal for controlling a component of the cardiac support system.

28. A cardiac support system comprising:
an inlet cannula for delivering blood volume flow to a flow pump;
an impeller of the flow pump; and
a device configured to monitor a state of wear of a cardiac support system, the device comprising:
a reading device configured to receive a sensor signal associated with a first operating state of the impeller of the cardiac support system;
a condition monitoring system to:
store a current fingerprint based at least in part on a sensor signal, compare the current fingerprint with a predetermined fingerprint based at least in part on a comparison rule;
determine a wear signal based at least in part on the comparison; and
determine a failure prediction associated with the cardiac support system based on the wear signal; and
cause an interface to provide an alert associated with the failure prediction.

29. The cardiac support system of claim 28, wherein the failure prediction includes a predicted time of failure of the cardiac support system.

30. The cardiac support system of claim 28, wherein the sensor signal is associated with deposits on the impeller or an imbalance of the impeller.

31. The cardiac support system of claim 28, wherein the sensor signal is associated with at least one of: position, vibration, acceleration, pressure, rotation rate, temperature, voltage, current, power, optical reflection, or electrical resistance associated with the impeller.

32. The cardiac support system of claim 28, wherein the sensor signal represents the operating state of the cardiac support system in the time domain or the frequency domain.

33. The cardiac support system of claim 28, wherein the control device is further to provide the sensor signal or the wear signal to an external processing device.
